SEQUENCE LISTING (6 sequences)

SEQ ID NO: 1 - DNA, 24 bp, Artificial
  Synthetic construct
cggccgcaattcgagctcggtacc

SEQ ID NO: 2 - DNA, 55 bp, Artificial
  Synthetic construct
gttggccgattcattaatgcagcagatcttgcggcacgacaggtttcccgactgg

SEQ ID NO: 3 - DNA, 36 bp, Artificial
  Synthetic construct
ctaatcgacctcgagcagatccgccaggcgtgtata

SEQ ID NO: 4 - DNA, 36 bp, Artificial
  Synthetic construct
tagaggatccccgggttacgccaagcttttcgaaac

SEQ ID NO: 5 - DNA, 45 bp, Artificial
  Synthetic construct
ccggatatcagatctaaaatggccctcccctcggacaaggccacc

SEQ ID NO: 6 - DNA, 20 bp, Artificial
  Synthetic construct
cccgaagcactctctgcccg

(19) United States
(12) Patent Application Publication
(10) Pub. No.: US 2013/0090471 A1
(43) Pub. Date: Apr. 11, 2013

(54) METHOD FOR DETECTING COMPOUNDS THAT MODULATE THE CHOLESTEROL METABOLISM

(71) Applicants: Bruno Dumas, Paris (FR); Isabelle Maury, Paris (FR)

(72) Inventors: Bruno Dumas, Paris (FR); Isabelle Maury, Paris (FR)

(73) Assignee: SANOFI, Paris (FR)

(21) Appl. No.: 13/702,825

(22) PCT Filed: Jun. 9, 2011
(86) PCT No.: PCT/FR2011/051316

§ 371 Date: Dec. 7, 2012

(30) Foreign Application Priority Data
Jun. 10, 2010 (FR) 1054566

(51) Int. Cl. C12Q 1/60

(52) U.S. Cl. CPC C12Q 1/60
USPC 544/178; 435/11; 435/320.1; 564/324; 564/306; 564/442

(57) ABSTRACT

The present invention relates to a method for identifying compounds that modulate cholesterol metabolism by detecting enzyme activities involved in the synthesis or catabolism of cholesterol. Said method enables the lethal risk for mammalian cells when the cholesterol level decreases to be avoided, due to the use of genetically modified yeasts, the survival of which is not strictly dependent upon the cholesterol production.

The subject of the present invention is a method for identifying compounds that modulate cholesterol, by detecting enzyme activities involved in the synthesis or catabolism of cholesterol. The subject of the present invention is also the use of the detected compounds in the treatment of cardiovascular or metabolic pathological conditions.

Cholesterol is the most important animal sterol. It is a fundamental component of cell membranes, the fluidity of which it controls, and is present in all animal tissues and particularly in the brain. Dysfunctions in terms of its synthesis and its catabolism are associated with numerous pathological conditions: atherosclerosis, angina pectoris, cardiovascular events, metabolic syndrome, diabetes, Smith-Lemli-Opitz syndrome, etc.

The detection of molecules capable of modulating the cholesterol level in the body is therefore an essential advance in combating all these pathological conditions.

Inhibitors of the synthesis or activators of the catabolism of cholesterol are known, such as triparanol which is an inhibitor of DHCR14/sterol-Δ14 reductase, DHCR24/sterol-Δ24 reductase, DHCR7/sterol-Δ7 reductase and Δ8-7 isomerase, AY9944 which is an inhibitor of DHCR7/sterol-Δ7 reductase (J. Sanchez-Wandelmer et al 2009), SR31747 which is an inhibitor of sterol Δ3-7 isomerase (R. Paul et al 1998), or amorolfine which is an inhibitor of DHCR14/sterol-Δ14 reductase and sterol Δ8-7 isomerase (A. J. Carrillo-Munoz et al; 2006).

However, the pathological conditions associated with cholesterol are numerous and require further progress in treatments and therefore new methods for studying and detecting potential modulators of cholesterol metabolism and transport.

The prior art describes the use of selection media in which increasing the cholesterol concentration leads to resistance to toxins, to antibiotics and to antifungal agents. Bringing a cholesterol-producing cell into contact with a cholesterol synthesis activator increases the amount of cholesterol in the cell. This leads to resistance to the toxin of the selection medium used, or to the antibiotic or antifungal agent used, and therefore growth of the cells (see European patent EP0727489 B1).

Similarly, an inhibitor of the enzymes for synthesizing cholesterol or an activator of the enzymes which catabolize cholesterol causes cell death. The disappearance of cholesterol is harmful to the life of the cell, in particular for the mammalian cells used up until now. This does not therefore enable the discovery of new modulators of cholesterol metabolism or transport.

A simple and effective method for detecting these modulators is therefore sought.

The applicant has shown, surprisingly, using yeasts, that it is possible to detect cholesterol modulators by means of media which enable inverse selection, wherein the cholesterol leads to less resistance to toxins. This makes it possible to determine cholesterol synthesis inhibitors and/or cholesterol catabolism activators when a resistance to the toxins is observed.

Advantageously, the cholesterol-producing yeast cells used in this test are genetically modified yeasts. This is because, in the natural state, yeasts produce ergosterol. The applicant uses genetically modified yeasts in order to divert the ergosterol production so as to produce cholesterol.

In one embodiment of the invention, the yeasts are modified as mentioned in patent EP 0727489 B1 or by deletion of the non-essential genes of the ERG family (ERG 6, ERG 5, ERG 4, ERG 2 and ERG 3).

One way to obtain yeasts of this type is also described in international patent application WO2005/121315 (Aventis Pharma).

The enzymes of the end of ergosterol or cholesterol synthesis and catabolism described in the present application are not essential to the wild-type yeast nor to the yeast modified to produce cholesterol; their activation or inhibition is therefore unrelated to the growth or death of these yeasts.

This therefore makes it possible to study cholesterol modulators without having any problem of survival of these cells: indeed, without cholesterol, the mammalian cells used up until now for tests of this type die. It is therefore impossible to observe the effect of compounds which reduce cholesterol synthesis since the cells die as soon as the cholesterol level is insufficient.

The present invention makes it possible to solve this problem through the use of genetically modified yeasts, wherein the survival of the cell is not strictly dependent upon its cholesterol production.

The invention which is the subject of the present application therefore provides a novel alternative to the means for detecting cholesterol synthesis modulators.

It consists in transforming yeasts genetically so that they produce both cholesterol and enzymes associated with cholesterol catabolism. The recombinant yeasts are brought into contact with a compound that potentially modulates cholesterol on a medium which is initially toxic for cholesterol-producing yeasts. The observation of the restoration of growth of the yeasts or, conversely, of death of the yeasts, makes it possible to determine whether the compound tested inhibits or increases cholesterol production by the yeasts.

The yeasts used may be Saccharomyces cerevisae, Schizosaccharomyces pombe, Kluyveromyces (lactis and the like) or Pichia pastoris.

Advantageously, the yeasts used are Saccharomyces cerevisae.

The compounds which induce a decrease in the amount of cholesterol produced either by inhibition of its synthesis or by activation of its catabolism confer restoration of growth of the cells on a medium which is initially toxic for these cells. Inhibitors of the enzymes involved in cholesterol synthesis and/or activators of cholesterol catabolism are thus demonstrated by means of this method for detecting cholesterol-level-dependent growth.

Such enzymes may be: DHCR14, DHCR24, DHCR7 and sterol Δ8-Δ7 isomerase for cholesterol synthesis.

Likewise, this method makes it possible to identify activators of enzymes which catabolize cholesterol, for instance cytochromes P450 (for example, CYP27A1 CYP46A1, CYP11A1 and CYP7A1).

The toxins according to the invention may be syringomycin E, phytosphingosine, telomycin, iturin A, Vibrio cholerae toxin and cholesterol-dependent toxins.

Advantageously, the toxin of the medium according to the invention is syringomycin E.

The yeast strains are transformed by introducing a vector which enables expression of genes encoding the production of enzymes associated with cholesterol catabolism.

Advantageously, the applicant uses a recombinant-protein expression vector optimised for the functional expression of cytochromes P450 and their electron-transporting cofactors.

In one embodiment of the invention, these recombinant vectors are plasmids.

The subject of the invention is illustrated, without, however, being limited thereto, by the following examples:

### Materials and Methods:

The medium used for culturing the yeasts is advantageously a Käppeli medium composed of 600 ml demineralized H₂O, 50 ml of 20-times concentrated salt solution (table 1), 10 g of casamino acids (Difco) or 8.9 g of (NH₄)₂SO₄ (Prolabo 21 333.296) (nitrogen source), 2 ml of 500-times concentrated vitamin solution (Mix 2), 0.2 ml of 5000-times concentrated solution of CaCl₂ and FeCl₃ (Mix 3) and 100 mg/L of amino acids and et 50 mg/L of required bases (except adenine at 100 mg/l) for 1 L medium in total.

The pH is adjusted to 5.5 with concentrated KOH, then the volume is adjusted to 900 ml before filtering through a 0.22 μm Nalgene filter and adding 100 ml of carbon source at 20% (D-glucose, Prolabo 24 370. 294).

| Mix 1: 20-times | | | | |
|---|---|---|---|---|
| concentrated | | | | |
| salt solution | Supplier | Reference | Concentration | |
| H₃PO₄ (85%) | PROLABO | 20 621.295 | 6.6 | ml/l |
| KH₂PO₄ | PROLABO | 26 936.293 | 57 | g/l |
| MgSO₄•7H₂O | PROLABO | 25 167.298 | 12 | g/l |
| Mn SO₄•H₂O | PROLABO | 25 300.290 | 0.32 | g/l |
| Cu SO₄•5H₂O | PROLABO | 23 174.290 | 8 | mg/l |
| Zn SO₄•7H₂O | PROLABO | 29 253.293 | 0.3 | g/l |
| Co Cl₂•6H₂O | PROLABO | 22 896.184 | 56 | mg/l |
| Na₂ MoO₄•2H₂O | PROLABO | 27 936.233 | 50 | mg/l |
| H₃BO₃ | PROLABO | 20 183.291 | 0.15 | g/l |
| Citric acid | PROLABO | 20 276.235 | 10 | mg/l |
| KI | PROLABO | 26 846.235 | 20 | mg |
| Ni SO₄•6H₂O | PROLABO | 25 897.197 | 50 | mg/l |
| Trisodium Citrate• | PROLABO | 27 833.237 | 0.5 | g/l |

2H₂O

The pH of this solution is between 2.6-2.7. The solution is stored at +4° C. and aliquoted as 50 ml per tube.

| Mix 2: 500-times | | | | |
|---|---|---|---|---|
| concentrated vitamin | | | | |
| solution | Supplier | Reference | Concentration | |
| Thiamine HCl (Vit. B1) | SIGMA | T-4625 | 1 | g/l |
| Pyridoxine HCl (Vit. B6) | SIGMA | P-9755 | 2.5 | g/l |
| Nicotinic acid | SIGMA | N-4126 | 4 | g/l |
| D-Biotin (Vit. H) | SIGMA | B-4501 | 25 | mg/l |
| Ca-D-Pantothenate | SIGMA | P-5710 | 5 | g/l |
| Meso-Inositol | SIGMA | I-5125 | 40 | g/l |

(Myo-Inositol)

The solution is stored at −20° C. and aliquoted as 2.2 ml per tube.

| Mix 3: 5000-times | | | Amount |
|---|---|---|---|
| concentrated | | | weighed out |
| solution | Supplier | Reference | (in g) |
| Ca Cl₂•2H₂O | PROLABO | 22 317.27 | 250 g/l |
| Fe Cl₃•7H₂O | PROLABO | 24 208.237 | 250 g/l |

The solution is stored at +4° C. and aliquoted as 500 μL per tube.

The sporulation media used for the yeasts are the following:

SP1 sporulation medium: yeast extract 2.5 g/L, potassium acetate 9.8 g/L, glucose 1 g/L, agar 20 g/L.

ACK sporulation medium: potassium acetate 10 g/L, agar 20 g/L.

### EXAMPLE A

### Construction of Plasmid Vectors

### Construction of Expression Vector for DHCR24 and Cytochrome P450

This plasmid was constructed for the purpose of obtaining, on one and the same vector, 3 expression cassettes for DHCR24 (dehydrocholesterol-24-reductase), mature CYP27A1 and the mature adrenodoxin (ADX) electron transporter, conferring in a modified yeast the synthesis of cholesterol and its catabolism via the activities of DHCR24 and CYP27A1, respectively. The activity of electron transporters such as ADX (provided on the plasmid) and ADR (carried on the genome of the strain) is necessary for functional activity of mitochondrial cytochrome P450 such as CYP11A1 (international patent application WO02/061109) or CYP27A1.

The plasmid pIM580 is obtained by the methods of molecular biology and recombination in yeast that are known to those skilled in the art. This expression vector derives from the plasmids pCD63 (Pompon et al 1998 Nat. Biotechnol.) and pYeDP60 (Pompon et al 1994 Eur. J. Biochem), and contains a 2μ origin of replication for S. cerevisiae and the URA3 selectable marker (FIG. 7).

To obtain this vector, the TRP1 selectable marker of pCD63 is deleted by homologous recombination in the yeast. For this, the fragment of pCD63 opened at the BsuI and SmaI restriction sites at the level of the sequence of the TRP1 selectable marker is brought into contact with the PCR fragment obtained with the primers of sequences SEQ ID Nos. 1 and 2 on pCD63 template, at the level of the BgIII sites hybridizing on either side of the TRP1 marker. This mixture of DNA fragments is then introduced into a yeast of W303 type by means of the LiAc/PEG transformation method described by Gietz et al; 1995 and 2002.

The recombinant clones are selected for the presence of the URA3 selectable marker and for the absence of the TRP1 marker by growth on uracil-free medium and lack of growth on tryptophan-free medium. The DNA is extracted from the yeasts by lysis with zymolyase (12.5 mg/ml, 1M Sorbitol, 0.1 M phosphate buffer, pH 7.2, 1 h at 37° C.), followed by alkaline lysis using the Qiagen kit, ref 12106, then transferred into TG1 bacteria (TSB method adapted by Chung et al 1989) for analysis using conventional molecular biology techniques. This new vector is called pIM565.

Next, the expression cassette for DHCR24 modified on the 2nd and 3rd codons, under the control of the TPI (Triose Phosphate Isomerase) promoter originating from the Nael fragment of pIM330 (derived from pYX212 #MBV-028-10 R&D systems) is introduced at the PvuII site of pIM565 so as to form the plasmid pIM578. The expression cassette for DHCR24 modified in terms of the 2nd and 3rd codons is described in international patent application WO2005/121315. The promoter chosen here is the TPI promoter, a constitutive promoter in place of CYC1.

Finally, the expression cassette for CYP11A1 (originating from pCD63) is replaced with that for mature CYP27A1 (i.e. devoid of the mitochondrion-targeting sequence) by homologous recombination in the yeast between the pIM578 vector opened at the Nael site and the PCR fragment derived from pIM558 with the primers of sequences SEQ ID Nos. 3 and 4 containing the expression cassette for mature CYP27A1 under the control of the Gal10/CYC1 chimeric promoter derived from pYeDP60 (D. Pompon et al; 1994 and 1996).

The cDNA of mature CYP27A1 originates from the clone with GenBank number NM_{—}000784, of which the N-terminal part devoid of the first 33 amino acids was modified by PCR with the primers of sequences SEQ ID Nos. 5 and 6.

The resulting plasmid containing the three expression cassettes for DHCR24, ADX et mature CYP27A1 is called pIM580.

The equivalent plasmid containing the coding sequence of CYP46A1 is called pIM584 and is obtained in a similar way. The cDNA of CYP46 derived from the clone with GenBank number NM_{—}006668 is introduced into pYeDP60 and then the expression cassette (promCYC1/GAL10-CYP46-TermPGK) is transferred into pIM578 by homologous recombination in the yeast.

The control plasmid devoid of coding sequence for cytochrome P450 is obtained in a similar manner by homologous recombination between the expression cassette for pYeDP60 and pIM578 opened at the Nael site. This control vector without P450 is called pIM582.

The plasmids which can be used for this invention are summarized in table 1 below:

TABLE 1

| Plasmid | P450 | DHCR24 | References |
|---|---|---|---|
| pCD63 | mature CYP11A1 | absent | Pompon et al 1998 |
| | | | Nat.Biotechnol. |
| pYeDP60 | none | absent | Pompon et al 1994 |
| | | | Eur.J.Biochem. |
| pIM565 | mature CYP11A1 | absent | The present application |
| pIM578 | mature CYP11A1 | present | The present application |
| pIM580 | mature CYP27A1 | present | The present application |
| pIM584 | CYP46A1 | present | The present application |
| pIM582 | none | present | The present application |

### EXAMPLE B

### Construction of the Modified Yeast Strains

In one advantageous embodiment, cholesterol-producing yeast strains were constructed as follows:

Construction of the yIM26 Strain:

The YIM126 strain was constructed to prevent the operating of genes involved in cholesterol modifications and in particular the two enzymes responsible for esterification with aliphatic chains ARE1 and ARE2 and the enzyme encoded by the ERG5 gene responsible for desaturation in position 22-23 of the side chain of ergostas and cholestas. These characteristics were combined in one and the same strain containing the elements required for cholesterol production.

For that, two haploid strains containing interesting characteristics are brought into contact so as to give a diploid strain which is subsequently sporulated by growth on a very rich and then very poor medium (as described in international patent application WO2005/121315). The mixture of the diploid cells and asci is then brought into contact with an aqueous medium containing 30% ether for 3 and 6 minutes in order to preferentially lyse the diploid strains. The surviving clones are then plated out on a selective medium according to the characteristics sought, in order to obtain a haploid strain combining the parental characters.

Thus, the yIM126 strain was obtained by selection of a haploid clone derived from three successive crosses of various strains using the methods known to those skilled in the art.

For that, the Fy11679-28c strain (international patent application WO2002/061109) is crossed with the ERT strain (WGIF01 strain of international patent application WO2005/121315). A diploid clone resulting from this cross, selected on the basis of the complementary autotrophies of the parental strains, is sporulated then treated as mentioned above for obtaining haploid clones. The haploid strains capable of growing on a medium devoid of adenine and tryptophan, the hallmark of the adenine autotrophic character of Fy1679-28c and the erg6:TRP1 disruption of ERT, were selected. The haploidy of the clones was verified by crossing with control strains W303 MATa or MATalpha. The resulting clones combining an intact ADE2 locus and disruption of the ERG6 gene by the selectable marker for tryptophan TRP1 were called yIM110 and yIM111.

Next, the yIM110 strain is crossed with the CDRO6 haploid strain. CDRO6 is a strain of genetic origin FY1679, the ade2 locus of which is non-functional through the integration of an expression cassette for Δ7-sterol reductase of the Arabidopsis thaliana plant. CDRO6 and CDRO7 (described in patent application WO2002/061109 or Duport et al; 2003) were obtained from the same cross and differ by virtue of the presence of the ERG5 gene which is functional in CDR06. A diploid clone yIM110XCDRO6 is isolated and then placed under spore production conditions. The spores are prepared as described previously and isolated on a rich medium and then tested on various media in the presence and absence of adenine and in the presence of nystatin in order to verify the resistance of the strain to this antifungal agent linked to the combination of the functional absence of the ERG6p activity and the presence of the Δ7-sterol reductase activity. Furthermore, the sterol composition is verified by saponification followed by organic extraction of the total sterols. The identity of the sterols is analyzed by GC/FID then verified by GC/MS. The presence of a product having the same retention time as desmosterol is observed for clones 4 and 6 using a pellet of cultured cells treated as previously described in international patent application WO2005/121315. These clones 4 and 6 are also resistant to nystatin when galactose is the carbon source. These haploid clones 4 and 6 are called, respectively, yIM115 and yIM116.

Finally, a 3rd cross with the yIM116 and CA23 strains is carried out for the purpose of introducing the interruption of three genes of interest, i.e. ARE1, ARE2 and ERG5, into a strain which can produce cholesterol, and also the expression cassette for an electron transporter required in order for cytochrome P450 to function. The CA23 strain described in international patent application WO2005/121315 contains a non-functional form of these 3 genes ARE1, ARE2 and ERG5 and the expression cassette for the adrenodoxin reductase (ADR) electron transporter, integrated at the LEU2 locus. A yIM116XCA23 diploid was isolated on a minimum medium containing neither tryptophan, histidine or leucine but containing adenine and uracil. In this way, only the diploid cells originating from a cross between yIM116 and CA23 can grow, whereas none of the partners of the cross can grow under these conditions. A diploid clone is sporulated under the conditions previously described. About a hundred spores are isolated after treatment with ether of a mixture of spores and of the diploid strain as previously described. These clones are characterized in terms of their mating-type sign, their capacities to grow on a medium free of adenine, leucine, histidine and tryptophan, and their capacities to withstand three antifungal agents, nystatin, hygromycin and geneticin. Finally, clone No. 4 thus selected was called yIM126. This clone can grow in the absence of leucine, indicating the presence of a functional LEU2 gene and therefore the presence of an expression cassette for the mature form of ADR under the control of the GAL10/CYC1 promoter. This clone can also grow in the absence of tryptophan and histidine, indicating the potential disruption of the ERG6 and ARE2 genes. It is resistant to high levels of nystatin and to geneticin, probably indicating the presence of the DHCR7 enzyme and also the disruption of the ARE1 gene, respectively. The free and esterified sterols are analyzed in order to confirm or refute the presence of the products of the ARE1 and ARE2 genes, and the quality of the sterols is analyzed in order to refute or confirm the interruption of the ERG6 gene and the presence of DHCR7.

TABLE 2
phenotype of the yeast strains used

| Strains | Genotype | References |
|---|---|---|
| yIM110-111 | Fy1679 − 28c × ERT = Fy Δer6 | this study |
| | Mat a/alpha, his3, leu2, trp1, ura3, erg6::TRP1, | |
| | nystatin R, Cycloheximide S | |
| yIM115-116 | yIM110 × CDR06 | this study |
| | MATa/alpha, his3, leu2, Δtrp1, ura3 | |
| | ade2::GAL10/CYC1-Δ7sterol-Reductase, | |
| | erg6::TRP1, Nystatin R, | |
| yIM126 | CA23 × yIM116: clone 4 | this study |
| | MATa, ura3, leu2, his3, trp1, erg6::TRP1, | |
| | ade2::prom GAL10/CYC1-Δ7sterol Reductase, | |
| | LEU2::promGAL10/CYC-bADRmat-termPGK | |
| | are2::HIS3, are1::Hygromycin, erg5::G418 | |

### EXAMPLE C

### Construction of a Strain Combining the Production of Cholesterol and its Catabolism Via Functional Enzyme Activities in the Yeast

The plasmids containing the expression cassettes for DHCR24 with or without cytochrome P450 and the ADX electron transporter are introduced into the yIM126 yeast using the LiAC/PEG transformation method (Gietz et al; 1995 and 2002).

The clones are selected on a uracil-free medium. Two to four clones of each combination are cultured for analysis of the total sterols by GC/FID then by GC/MS according to the procedure described in international patent application WO2005/121315. The clones are cultured for 48 h with shaking at 30° C. in YBN medium supplemented with 2% glucose and adenine at 100 μg/ml.

The optical density (OD600 nm) reaches an average value of 7 units. The cell pellets from a culture volume V are harvested and transferred into an identical volume V of Käppeli medium containing 20% casamino acids, 2% glucose and 100 μg/ml adenine. These cultures are incubated for 72 h to 90 h at 30° C. and reach an optical density at 600 nm of from 40 to 50 units. The pellet equivalent to a volume of 100 optical density units is treated for analysis by gas chromatography as previously mentioned and described in international patent application WO2005/121315.

The sterol profiles of the yeast samples are compared with the retention times of reference solutions by GC/FID gas chromatography, such as cholesterol (FIG. 1A), desmosterol (FIG. 1B), pregnenolone (FIG. 10), 24OH-cholesterol (FIG. 1D) and 27OH-cholesterol (FIG. 1E), and the products are identified by retention time similarity.

The analyses and the measurement of the surface area of the peaks show cholesterol production for the yIM126 strain containing the pIM580 plasmid without P450, with a cholesterol/desmosterol ratio greater than 3. The yIM126 strains containing the expression constructs for P450 produce a cholesterol derivative specific for each P450 which uses cholesterol as substrate: i.e. production of pregnenolone for CYP11A1 (FIG. 3), of 27OH-cholesterol and of 27OH-sterols for CYP27A1 (FIG. 4) and of 24OH-cholesterol for CYP46A1 (FIG. 5). The cholesterol/desmosterol ratio decreases by a factor of 2 in the presence of CYP46 and by a factor of 5 to 6 in the presence of CYP11A1 or CYP27A1, indicating that cholesterol is probably metabolized. New sterols hydroxylated in position 27, such as 27OH-desmosterol, appear specifically in the presence of the CYP27A1 activity (FIG. 4).

### EXAMPLE D

### Biological Method in Cellulo for Detecting Cholesterol Metabolism

### a) Culture Medium:

A culture medium is developed which makes it possible, through a simple phenotypic test for growth or for lack of growth, to distinguish the cholesterol-producing yeasts from those which do not produce cholesterol or which catabolize it. This culture medium is a rich medium of YPG agar type (YPG complete medium: yeast extract (Difco) 10 g/L, bactopeptone (Difco) 20 g/L, glucose (Merck) 20 g/L) supplemented with syringomycin E toxin from Pseudomonas syringae B-301D (Sigma #S6946) from 150 to 250 ng/ml.

Agar (20 g/L) is added in order to obtain solid media.

### b) Detection of Cholesterol Metabolism

The yIM126 yeast strains containing an expression vector for DHCR24 and cytochrome P450 such as pIM580, described previously, are cultured for 48 h with shaking at 30° C. in a YNB medium (yeast nitrogen base w/o amino acids (Difco) 6.7 g/L, glucose (Merck) 20 g/L) supplemented with 2% glucose and adenine at 100 μg/ml.

The culture reaches an optical density at 600 nm of about 7 units. The cell pellet is harvested by centrifugation and transferred into an identical volume of Käppeli medium containing 20% casamino acids, 2% glucose and 100 μg/ml of adenine, and shaken at 30° C. for 24 h, reaching an optical density at 600 nm of about 40 units.

The cultures are diluted to 1 unit of optical density at 600 nm and then to 1/50 and then 25-fold dilutions in order to perform a drop test on YPG agars containing syringomycin E according to the methods known to those skilled in the art.

After incubation for 48 h to 72 h at 30° C., the yeasts containing the pIM582 expression vector (without P450, FIG. 6, column 1) producing cholesterol are not capable of growing, whereas the yeasts containing the pIM580 expression vector (with CYP27A1, FIG. 6, column 2) producing cholesterol and converting it into derived products are capable of growing on a YPG agar containing syringomycin E at 170 ng/ml (FIGS. 6A and 6B). The toxicity of this YPG medium containing syringomycin E depends on the amount of cholesterol available in the yeast, observed by GC/FID or GC/MS.

### EXAMPLE E

### Biological Method in Cellulo for Detecting a Product which Interferes with Cholesterol Metabolism

This test for growth of yeasts modified with respect to cholesterol synthesis also makes it possible to identify products which interfere either with cholesterol synthesis or with cholesterol catabolism.

The yIM126 yeasts containing the expression plasmids for DHCR24 with or without P450 are cultured in YNB medium, 2% glucose, adenine at 100 μg/ml for 48 h at 30° C. and then in Käppeli medium for 24 h at 30° C. as described above.

The yeast suspensions are diluted to an optical density at 600 nm of 0.005 unit ( 1/200) in YNB medium in order to inoculate a YPG agar containing syringomycin E at a dose that is toxic for the strain, i.e. 160 ng/ml for yIM126 containing the pIM582 plasmid, or 250 ng/ml for the yIM126 strain containing the pIM580 plasmid.

The inoculation of the agar medium is carried out by flooding its surface and immediately suctioning off the excess liquid. Products of interest are deposited on the agar surface thus inoculated, and then dried. After incubation for 48 h to 72 h at 30° C., yeast growth halos are observed around the deposits of the products which interfere with cholesterol synthesis or cholesterol catabolism. The products which induce the decrease in the amount of cholesterol either by inhibition of its synthesis or by activation of its catabolism confer restoration of growth on this medium which is initially toxic. Inhibitors of the enzymes involved in cholesterol synthesis, such as DHCR24, DHCR7 or Δ8-Δ7sterol isomerase, and which are not essential to the wild-type yeast or to a yeast modified with respect to sterol synthesis, are thus easily demonstrated by means of this method for detecting cholesterol-level-dependent growth. Similarly, this method makes it possible to identify activators of enzymes, such as cytochromes P450, for instance CYP27A1, which catabolize cholesterol. This is verified with known products, for instance triparanol which inhibits sterol-Δ14 reductase, DHCR7 and Δ8-7 isomerase, AY9944 which inhibits DHCR7 (J. Sanchez-Wandelmer et al 2009), SR31747 which inhibits sterol Δ8-7 isomerase (R. Paul et al 1998), or amorolfine which inhibits DHCR14 and sterol Δ8-7 isomerase (A. J. Carrillo-Munoz et al; 2006); likewise, the deposit of cholesterol or of 27OH-cholesterol induces an area of protection with respect to the toxicity of the syringomycin E contained in the agar (FIG. 6).

### FIGURE LEGEND

FIG. 1A: retention time of cholesterol by GC/FID gas chromatography.

FIG. 1B: retention time of desmosterol by GC/FID gas chromatography.

FIG. 1C: retention time of pregnenolone by GC/FID gas chromatography.

FIG. 1D: retention time of 24OH-cholesterol by GC/FID gas chromatography.

FIG. 1E: retention time of 27OH-cholesterol by GC/FID gas chromatography.

FIG. 2: The sterol profile of the yIM126 strain in the absence of P450 activity shows the predominant presence of cholesterol (RT 21.98 min) and of desmosterol (Rt 23.11 min).

FIG. 3: In the presence of CYP11A1 activity, the sterol profile of the yIM126 strain is modified with a decrease in the ratio of cholesterol (RT 21.97) to the benefit of desmosterol (RT 23.13), with the appearance of a sterol corresponding to pregnenolone (RT 14.73 min).

FIG. 4: In the presence of CYP27A1 activity, the sterol profile of the yIM126 strain is modified with a decrease in the ratio of cholesterol (RT 21.96) to the benefit of desmosterol (RT 23.11), and the appearance of 2 other sterols corresponding to 27OH-cholesterol (RT 33.53 min) and 27OH-desmosterol (RT 33.93 min).

FIG. 5: In the presence of CYP46A1 activity, the sterol profile of the yIM126 strain is modified, with the appearance of a sterol corresponding to 24OH-cholesterol (RT 30.38 min).

FIG. 6: Test for growth of various strains producing and/or catabolizing cholesterol on a YPG agar containing syringomycin E (SRG): Yeast culture dilutions deposited on a YPG agar without syringomycin E(6A) or with syringomycin E at 170 ng/ml (6B); Dilution OD at 600 nm from top to bottom: 0.02/0.0008/00003.

FIG. 7: Test for restoration of growth on a YPG agar containing syringomycin E inoculated with a cholesterol-producing strain.

The surface of a YPG agar medium containing syringomycin E at 250 ng/ml is inoculated with a covering of yIM126-pIM580-CYP27A1 yeast suspension. 2 μl of products at 1 mM or 0.1 μg/ml are deposited on the dried and inoculated surface.

FIG. 7A from top to bottom: Cholesterol, 27OH-cholesterol and amorolfine at 0.1 mg/ml.

FIG. 7B from top to bottom: Triparanol, AY9944, SR31747 at 1 mM.

FIG. 8: map of pIM580 plasmid.

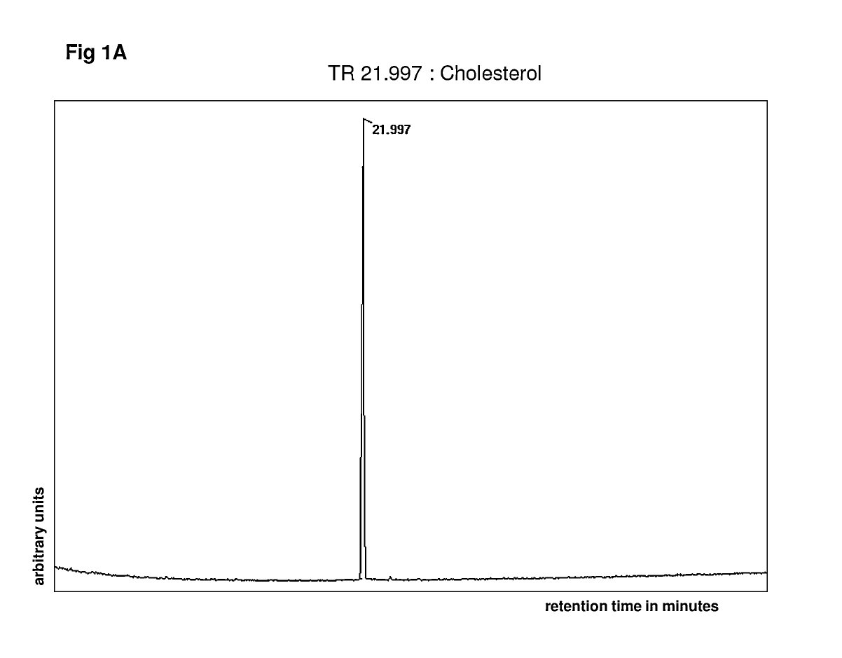

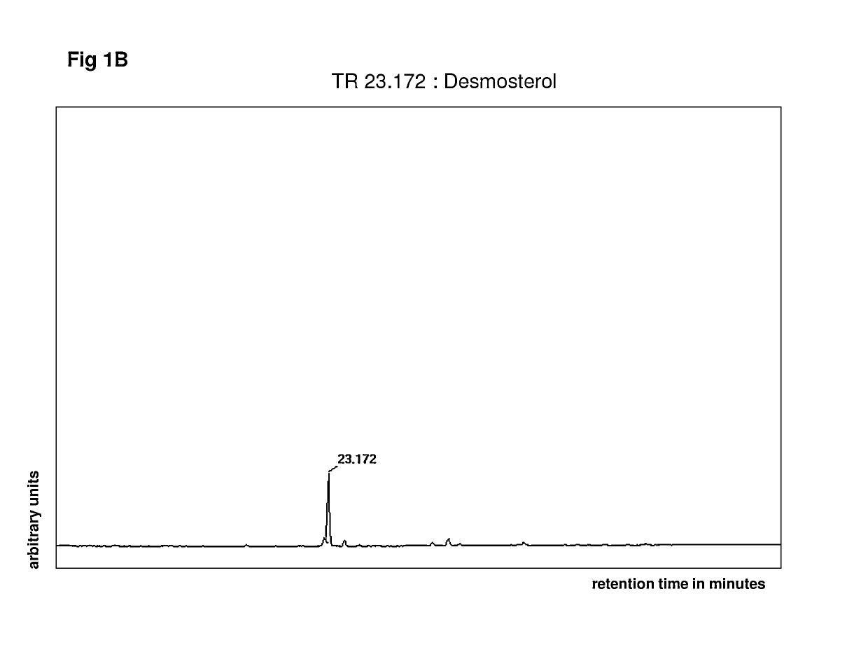

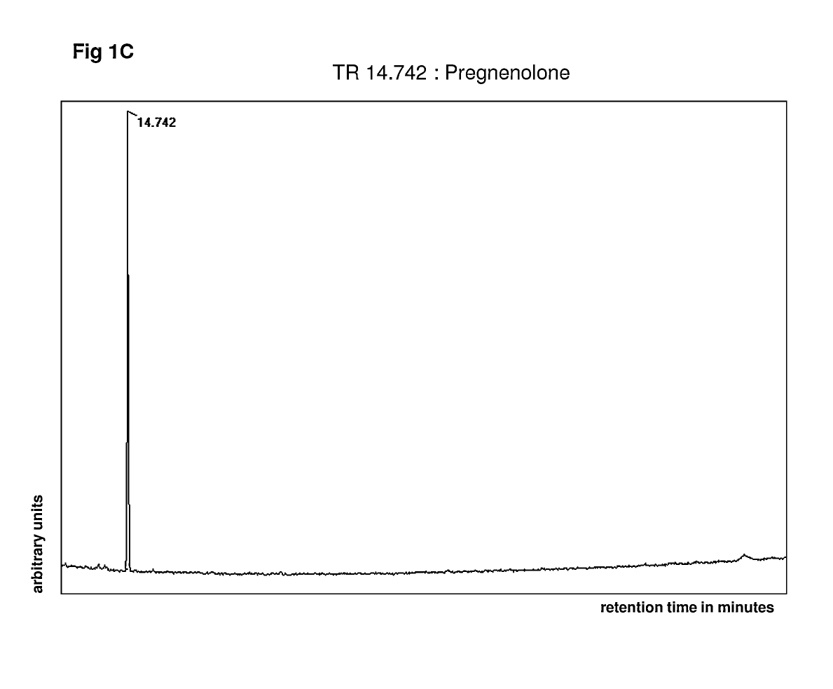

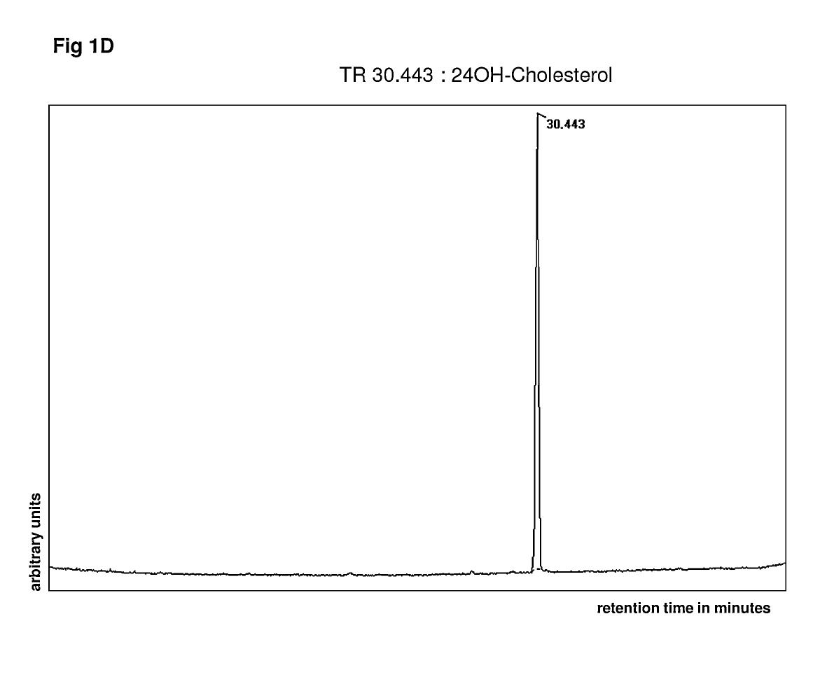

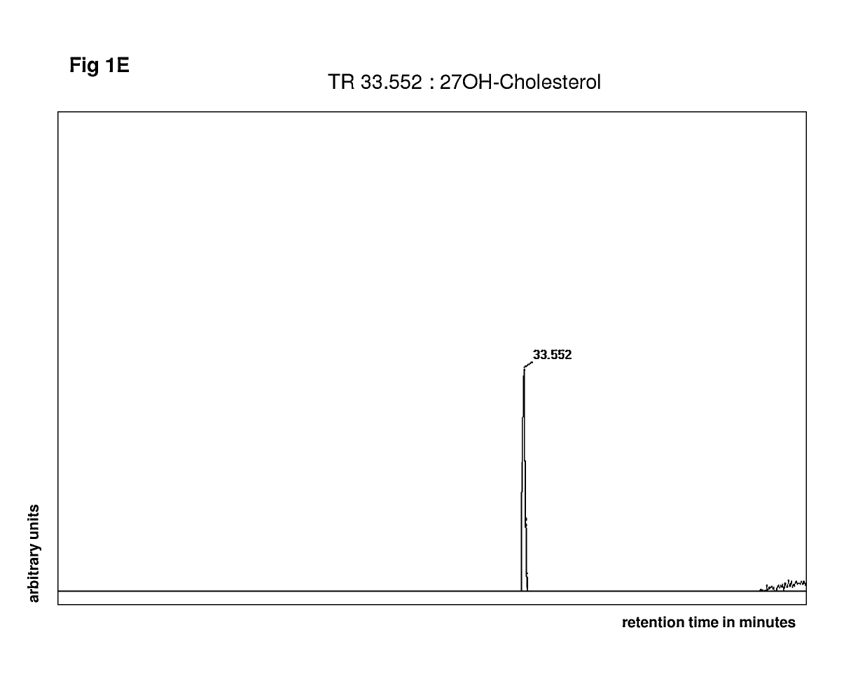

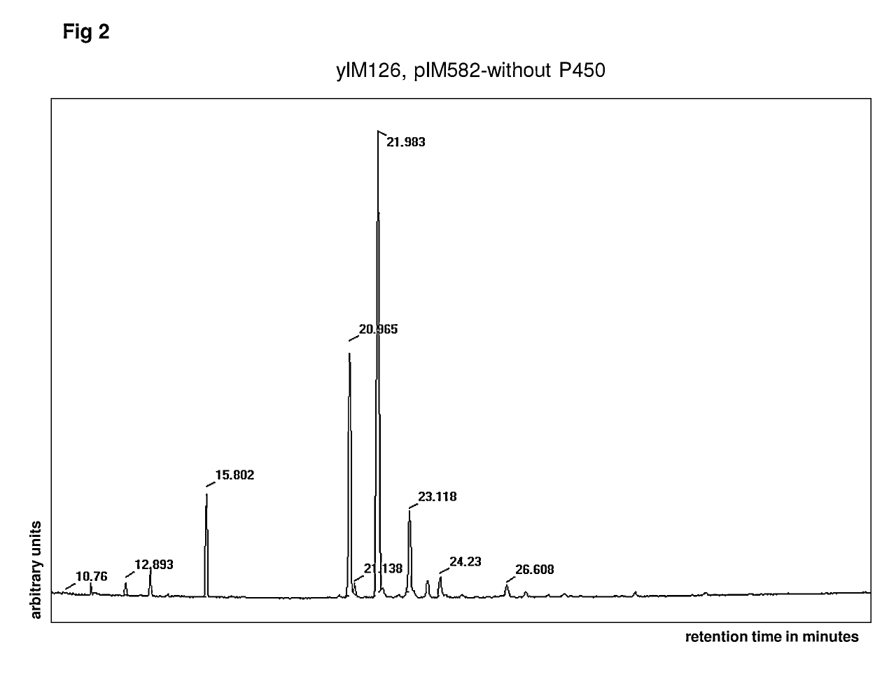

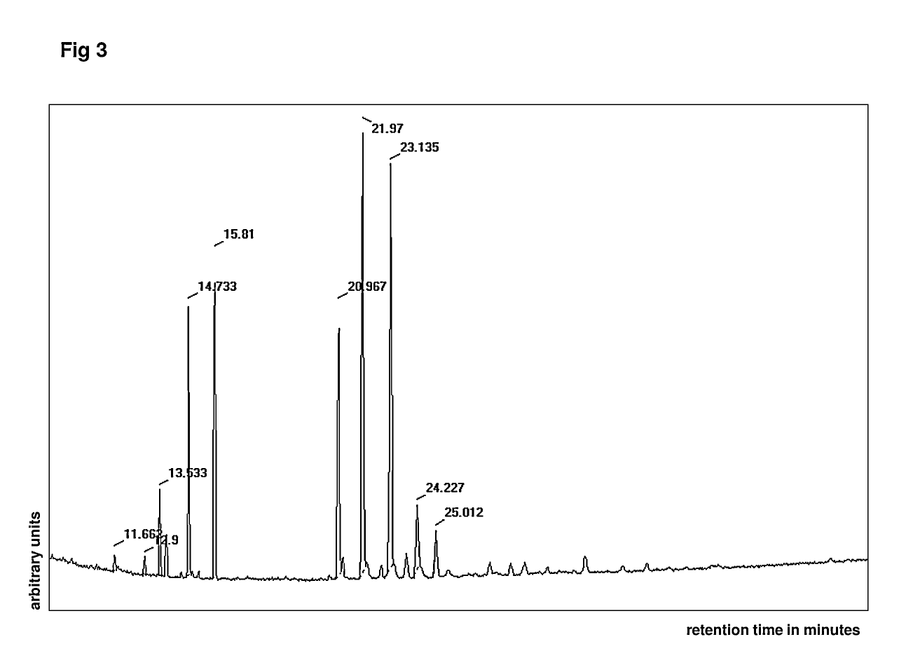

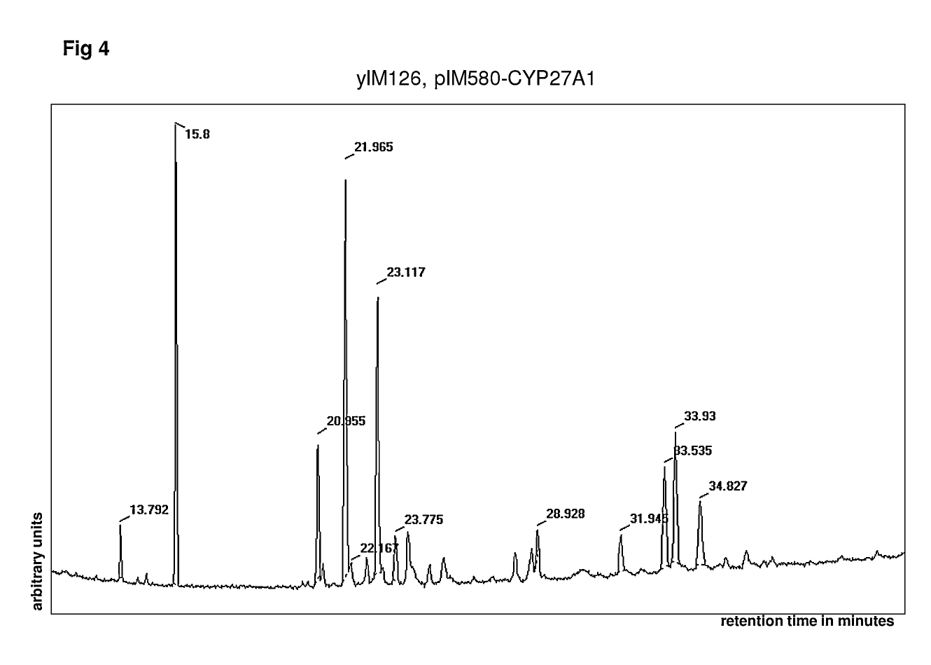

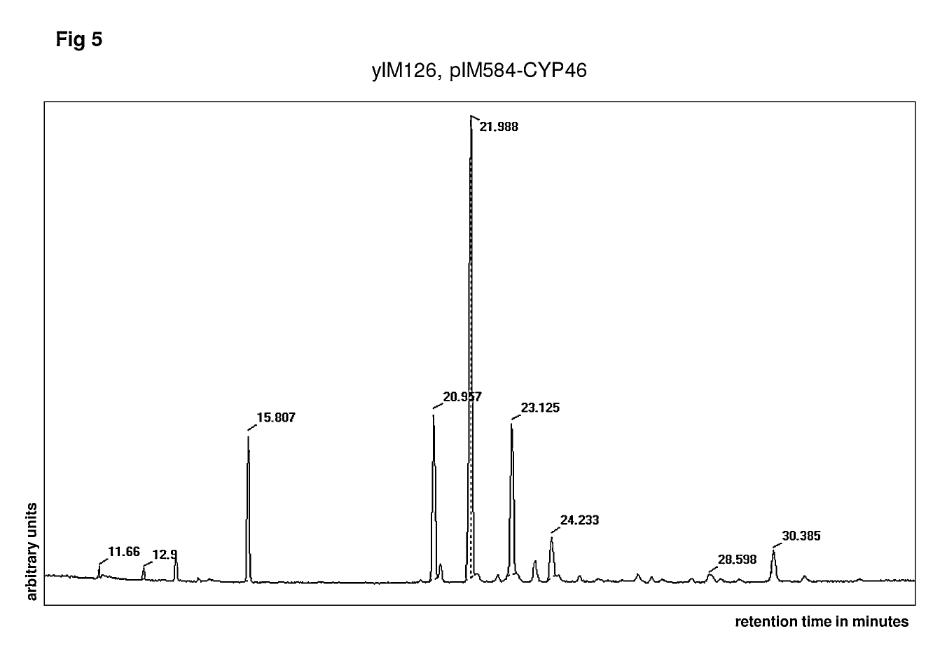

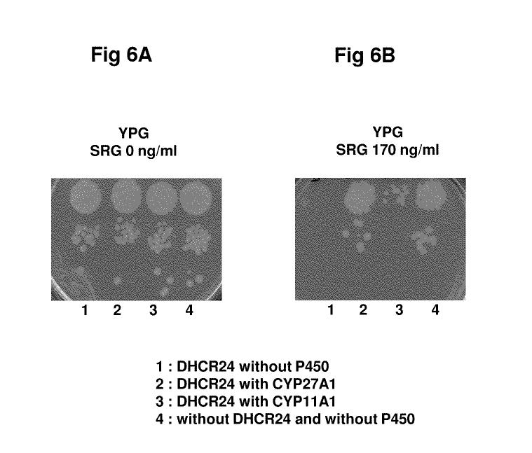

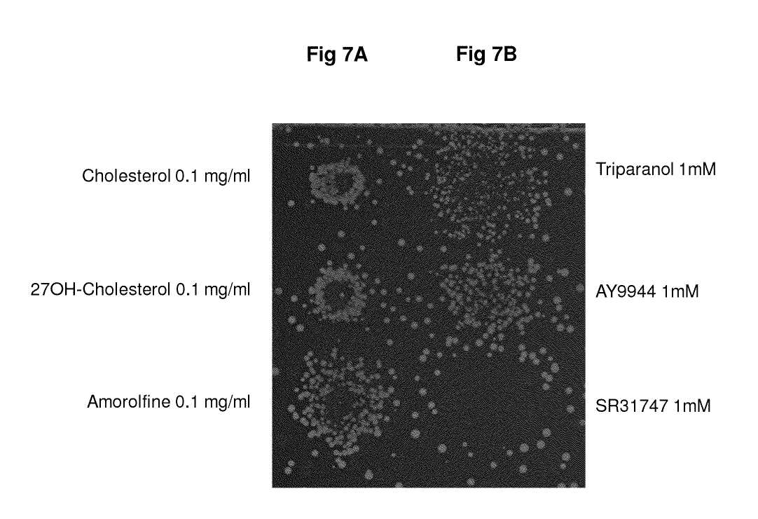

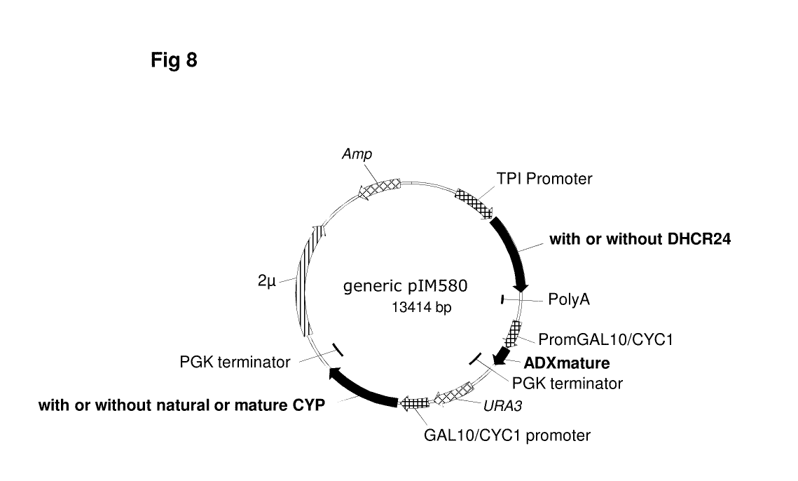

### LITERATURE

Carrillo-Munoz, A. J., G. Giusiano, P. A. Ezkurra, and G. Quindos. 2006. Antifungal agents: mode of action in yeast cells. Rev Esp Quimioter. 19:130-9.
Duport, C., R. Spagnoli, E. Degryse, and D. Pompon. 1998. Self-sufficient biosynthesis of pregnenolone and progesterone in engineered yeast. Nat. Biotechnol. 16:186-9.
Gietz, R. D., R. H. Schiestl, A. R. Willems, and R. A. Woods. 1995. Studies on the transformation of intact yeast cells by the LiAc/SS-DNA/PEG procedure. Yeast. 11:355-60.
Gietz, R. D., and R. A. Woods. 2002. Transformation of yeast by lithium acetate/single-stranded carrier DNA/polyethylene glycol method. Methods Enzymol. 350:87-96.
Paul, R., S. Silve, N. De Nys, P. H. Dupuy, C. L. Bouteiller, J. Rosenfeld, P. Ferrara, G. Le Fur, P. Casellas, and G. Loison. 1998. Both the immunosuppressant SR31747 and the antiestrogen tamoxifen bind to an emopamil-insensitive site of mammalian Delta8-Delta7 sterol isomerase. J Pharmacol Exp Ther. 285:1296-302.
Pompon, D., B. Louerat, A. Bronine, and P. Urban. 1996. Yeast expression of animal and plant P450s in optimized redox environments. Methods Enzymol. 272:51-64.
Sanchez-Wandelmer, J., A. Davalos, E. Herrera, M. Giera, S. Cano, G. de la Pena, M. A. Lasuncion, and R. Busto. 2009. Inhibition of cholesterol biosynthesis disrupts lipid raft/caveolae and affects insulin receptor activation in 3T3-L1 preadipocytes. Biochim Biophys Acta. 1788:1731-9.
P. Urban, D. Werck-Reichhart, H. G. Teutsh, F. Durst, S. Regnier, M. Kazmaier and D. Pompon, Characterization of recombinant plant cinnamate 4-hydroxylase produced in yeast Kinetic and spectral properties of the major plant P450 of the phenylpropanoid pathway, Eur J. Biochem. 1994 Jun. 15; 222(3): 843-850.

1. A method for identifying compounds that modulate cholesterol, characterized in that it comprises a cholesterol detection step and that the cholesterol level detected is inversely proportional to the growth of cells in a toxic medium.

2. The method as claimed in claim 1, characterized in that it comprises bringing a cholesterol-producing cell and a compound of interest into contact on a medium which is initially toxic for said cell.

3. The method as claimed in claim 1, characterized in that it comprises bringing a cell which produces both cholesterol and an enzyme that participates in cholesterol synthesis, and a compound of interest into contact on a medium which is initially toxic for said cell.

4. The method as claimed in claim 1, characterized in that it comprises bringing a cell which produces both cholesterol and an enzyme that catabolizes cholesterol, and a compound of interest into contact on a medium which is initially toxic for said cell.

5. The method as claimed in claim 1, characterized in that it comprises the use of cholesterol-producing genetically modified yeasts.

6. The method as claimed in claim 1, characterized in that it comprises the use of yeasts included in the group consisting of: Saccharomyces cerevisae, Schizosaccharomyces pombe, Kluyveromyces (lactis and the like) or Pichia pastoris, said yeasts being genetically modified so as to produce cholesterol.

7. The method as claimed in claim 1, characterized in that it comprises the following steps:
(a) transforming a yeast so that it expresses both cholesterol and an enzyme which activates cholesterol production,
(b) bringing the transformed yeast into contact with a compound of interest, and
(c) quantifying the change in the level of cholesterol expressed by means of a test for resistance to a toxin.

8. The method as claimed in claim 1, characterized in that it comprises the following steps:
(a) transforming a yeast so that it expresses both cholesterol and an enzyme which decreases cholesterol production,
(b) bringing the transformed yeast into contact with a compound of interest, and
(c) quantifying the change in the level of cholesterol expressed by means of a test for resistance to a toxin.

9. The method as claimed in claim 7, characterized in that the toxin used is included in the group consisting of syringomycin E, phytosphingosine, telomycin, iturin A, Vibrio cholerae toxin and cholesterol-dependent toxins.

10. The method as claimed in claim 9, characterized in that the toxin used is syringomycin E.

11. The method as claimed in claim 7, characterized in that the enzyme produced by the genetically modified yeast is chosen from the following group: DHCR7, DHCR14, DHCR24 and Δ8-7 sterol isomerase.

12. The method as claimed in claim 7, characterized in that the genetically modified yeast produces a cytochrome P450 chosen from the group consisting of CYP27A1, CYP46A1, CYP11A1 and CYP7A1 as an enzyme activator.

13. A vector comprising enzyme expression cassettes for enabling the conversion of yeast sterols to cholesterol and an expression cassette for a protein activity which acts on cholesterol metabolism.

14. The vector as claimed in claim 13, characterized in that it is a plasmid.

15. The vector as claimed in claim 13, characterized in that it is one of the following plasmids: pCD63, pYeDP60, pIM565, pIM578, pIM580, pIM582 or pIM584.

16. A compound detected according to the method described in claim 1, for the use thereof in the treatment of cardiovascular pathological conditions or of metabolic disorders.

17. The method as claimed in claim 8, characterized in that the toxin used is included in the group consisting of syringomycin E, phytosphingosine, telomycin, iturin A, Vibrio cholerae toxin and cholesterol-dependent toxins.

18. The method as claimed in claim 17, characterized in that the toxin used is syringomycin E.

19. The method as claimed in claim 8, characterized in that the enzyme produced by the genetically modified yeast is chosen from the following group: DHCR7, DHCR14, DHCR24 and 48-7 sterol isomerase.

20. The method as claimed in claim 8, characterized in that the genetically modified yeast produces a cytochrome P450 chosen from the group consisting of CYP27A1, CYP46A1, CYP11A1 and CYP7A1 as an enzyme activator.
